(19) United States
(12) Patent Application Publication
(10) Pub. No.: US 2011/0287204 A1
(43) Pub. Date: Nov. 24, 2011

(54) MANUFACTURE OF ETHYLENE/CARBOXYLIC ACID VINYL ESTER COPOLYMERS FROM RENEWABLE MATERIALS, COPOLYMERS OBTAINED AND USES

(75) Inventors: Samuel Devisme, Rouen (FR); Fabrice R. Chopinez, Bala Cynwyd, PA (US); Christian Laurichesse, Lons (FR); Thomas Roussel, Lyon (FR); Jean-Luc Dubois, Millery (FR)

(73) Assignee: ARKEMA FRANCE, Colombes (FR)

(21) Appl. No.: 13/129,155

(22) PCT Filed: Nov. 10, 2009
(86) PCT No.: PCT/FR2009/052165

§ 371 Date: Aug. 11, 2011

(30) Foreign Application Priority Data
Nov. 13, 2008 (FR) 0857686

(51) Int. Cl. B32B 27/28; C08L 31/02; C08F 218/02; B32B 1/08; C12P 7/62

(52) U.S. Cl. 428/36.9; 428/35.7; 442/327; 442/181; 435/135; 526/331; 524/563

(57) ABSTRACT

The present application relates to a process for the manufacture of copolymers of ethylene and of at least one vinyl ester, comprising the following steps: a) fermentation of renewable starting materials so as to produce at least one alcohol comprising ethanol; b) dehydration of the alcohol obtained so as to produce at least one alkene comprising ethylene and, optionally, purification of the alkene so as to obtain ethylene, c) copolymerization of the ethylene with at least one vinyl ester, d) isolation of the copolymer obtained. The application also relates to the copolymers of ethylene and of at least one vinyl ester in which the ethylene is at least partly obtained from renewable starting materials, and to uses thereof.

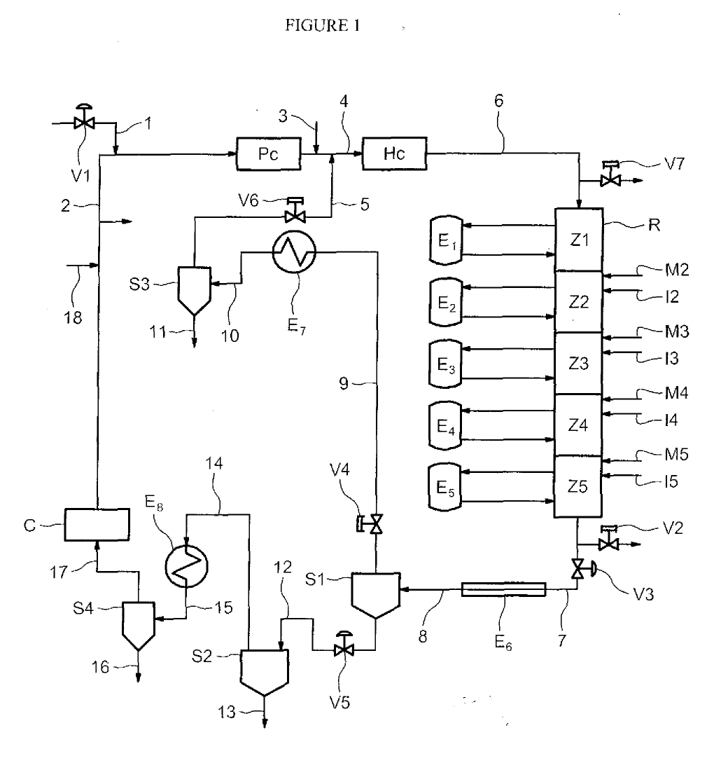

### FIELD OF THE INVENTION

The present invention relates to copolymers of ethylene and of at least one vinyl ester, in which the ethylene is at least partially obtained from renewable starting materials.

### STATE OF THE ART

One of the problems posed by copolymers comprising ethylene of the prior art is that they are produced starting from nonrenewable starting materials of fossil (oil) origin. In point of fact, oil resources are limited and the extraction of oil requires drilling to increasingly deep depths and under technical conditions which are ever more difficult, requiring sophisticated equipment and use of processes which are ever more expensive in energy. These constraints have a direct consequence with regard to the cost of manufacture of ethylene and thus of ethylene-based copolymers.

Advantageously and surprisingly, the inventors of the present patent application have employed a process for the industrial manufacture of ethylene-based copolymers from renewable starting materials.

The process according to the invention makes it possible to dispense, at least in part, with starting materials of fossil origin and to replace them with renewable starting materials.

In addition, the ethylene-based copolymers obtained according to the process according to the invention are of a quality such that they can be used in all the applications in which the use of these copolymers is known, including in applications with the highest standards.

### SUMMARY OF THE INVENTION

A subject matter of the invention is copolymers of ethylene and of at least one carboxylic acid vinyl ester, in which the ethylene is at least partially obtained from renewable starting materials.

In the present patent application, reference is made to compounds comprising carbon atoms “obtained from renewable starting materials”; thus, within the meaning of the present patent application, it will be understood that these compounds comprise ¹⁴C carbon atoms which can be determined according to the standard ASTM D 6866-06. For example, the copolymer can comprise at least 0.24×10⁻¹⁰% by weight of ¹⁴C.

Within the meaning of the present patent application, the term “copolymers of ethylene and of at least one vinyl ester” will be understood to mean both the copolymers consisting of two monomers and the terpolymers comprising ethylene and at least one vinyl ester.

Advantageously, the vinyl esters used in the copolymers according to the present invention are vinyl esters of C₂-C₈ carboxylic acids; preferably, they are chosen from vinyl esters of C₂-C₄ carboxylic acids and more preferably still they are chosen from vinyl acetate and vinyl propionate.

According to a specific alternative form, at least a portion of the carbon atoms of the vinyl ester is of renewable origin.

The copolymers according to the present invention can also be terpolymers of ethylene, of at least one vinyl ester and of at least one unsaturated carboxylic acid anhydride, in which terpolymers the ethylene is at least partially obtained from renewable starting materials and, optionally, at least a portion of the carbon atoms of the vinyl ester and/or at least a portion of the carbon atoms of the unsaturated carboxylic acid anhydride are of renewable origin.

Thus, according to a first specific alternative form of these terpolymers, at least a portion of the carbon atoms of the vinyl ester is of renewable origin.

According to a second specific alternative form of these terpolymers, at least a portion of the carbon atoms of the unsaturated carboxylic acid anhydride is of renewable origin.

According to a third specific alternative form of these terpolymers, at least a portion of the carbon atoms of the vinyl ester is of renewable origin and at least a portion of the carbon atoms of the unsaturated carboxylic acid anhydride is of renewable origin.

Preferably, the vinyl ester is chosen from vinyl acetate and vinyl propionate.

Preferably, the unsaturated carboxylic acid anhydride is maleic anhydride.

Thus, according to a first specific alternative form of these terpolymers, at least a portion of the carbon atoms of the vinyl ester is of renewable origin.

According to a second specific alternative form of these terpolymers, at least a portion of the carbon atoms of the unsaturated carboxylic acid anhydride is of renewable origin.

The copolymers according to the present invention can also be terpolymers of ethylene, of at least one vinyl ester, such as vinyl acetate, and of at least one unsaturated carboxylic acid ester, such as a carboxylic acid acrylic or methacrylic ester, in which the ethylene is at least partially obtained from renewable starting materials and, optionally, at least a portion of the carbon atoms of the vinyl ester and/or at least a portion of the carbon atoms of the unsaturated carboxylic acid ester are of renewable origin.

The present patent application also relates to the blends of copolymers according to the invention, to the compositions comprising these copolymers and to the uses of these copolymers.

The copolymers of ethylene and of at least one vinyl ester according to the present patent application are capable of being obtained according to the manufacturing process comprising the following stages:
a) fermentation of renewable starting materials and optionally purification, in order to produce at least one alcohol chosen from ethanol and mixtures of alcohols comprising ethanol;
b) dehydration of the alcohol obtained in order to produce at least one alkene chosen from ethylene and mixtures of alkenes comprising ethylene and optionally purification of the alkene in order to obtain ethylene,
c) copolymerization of the ethylene with at least one comonomer chosen from carboxylic acid vinyl esters,
d) isolation of the copolymer obtained.

The terpolymers of ethylene, of at least one vinyl ester and of at least one unsaturated carboxylic acid anhydride according to the present patent application are capable of being obtained according to the manufacturing process described above in which stage c) is a stage of copolymerization of the ethylene with at least one vinyl ester and at least one unsaturated carboxylic acid anhydride.

Other subject matters, aspects or characteristics of the invention will become apparent on reading the following description.

### DETAILED DESCRIPTION OF THE INVENTION

Stage a) of the process for the manufacture of copolymers of ethylene and of at least one vinyl ester according to the invention comprises the fermentation of renewable starting materials in order to produce at least one alcohol, said alcohol being chosen from ethanol and mixtures of alcohols comprising ethanol.

A renewable starting material is a natural resource, for example animal or plant, the stock of which can be built up again over a short period on the human scale. In particular, it is necessary for this stock to be able to be renewed as quickly as it is consumed. For example, plant materials exhibit the advantage of being able to be cultivated without their consumption resulting in an apparent reduction in natural resources.

Unlike the materials resulting from fossil materials, renewable starting materials comprise ¹⁴C. All the samples of carbon drawn from living organisms (animal or plant) are in fact a mixture of 3 isotopes: ¹²C (representing approximately 98.892%), ¹³C (approximately 1.108%) and ¹⁴C (traces: 1.2×10⁻¹⁰%). The ¹⁴C/¹²C ratio of living tissues is identical to that of the atmosphere. In the environment, ¹⁴C exists in two predominant forms: in the form of carbon dioxide gas (CO₂) and in organic form, that is to say in the form of carbon incorporated in organic molecules.

In a living organism, the ¹⁴C/¹²C ratio is kept constant by the metabolism because the carbon is continually exchanged with the external environment. As the proportion of ¹⁴C in the atmosphere is constant, it is the same in the organism as long as it is alive, since it absorbs this ¹⁴C in the same way as the ambient ¹²C. The mean ¹⁴C/¹²C ratio is equal to 1.2×10⁻¹².

¹²C is stable, that is to say that the number of ¹²C atoms in a given sample is constant over time. ¹⁴C is radioactive; the number of ¹⁴C atoms in a sample decreases over time (t), its half life being equal to 5730 years.

The ¹⁴C content is substantially constant from the extraction of the renewable starting materials up to the manufacture of the copolymer according to the invention and even up to the end of the lifetime of the object manufactured in said copolymer.

Consequently, the presence of ¹⁴C in a material, whatever the amount thereof, gives an indication with regard to the origin of the molecules constituting it, namely whether they originate from renewable starting materials and not from fossil materials.

The amount of ¹⁴C in a material can be determined by one of the methods described in the standard ASTM D 6866-06 (Standard Test Methods for Determining the Biobased Content of Natural Range Materials Using Radiocarbon and Isotope Ratio Mass Spectrometry Analysis).

This standard comprises three methods of measuring the organic carbon resulting from renewable starting materials, referred to as “biobased carbon”. The proportions indicated for the copolymer of the invention are preferably measured according to the mass spectrometry method or the liquid scintillation spectrometry method described in this standard and very preferably by mass spectrometry.

These measurement methods evaluate the ratio of the ¹⁴C/¹²C isotopes in the sample and compare it with a ratio of the ¹⁴C/¹²C isotopes in a material of biological origin giving the 100% standard, in order to measure the percentage of organic carbon in the sample.

Preferably, the copolymer according to the invention comprises an amount of carbon resulting from renewable starting materials of greater than 20% by weight, preferably of greater than 50% by weight, with respect to the total weight of carbon of the copolymer.

In other words, the copolymer can comprise at least 0.24×10⁻¹⁰% by weight of ¹⁴C and preferably at least 0.6×10⁻¹⁰% by weight of ¹⁴C.

Advantageously, the amount of carbon resulting from renewable starting materials is greater than 75% by weight, preferably equal to 100% by weight, with respect to the total weight of carbon in the copolymer.

Use may be made, as renewable starting materials, of plant materials, materials of animal origin or materials of plant or animal origin resulting from recovered materials (recycled materials).

Within the meaning of the invention, the materials of plant origin comprise at least sugars and/or starches.

The plant materials comprising sugars are essentially sugar cane and sugar beet; mention may also be made of maple, date palm, sugar palm, sorghum or American agave; the plant materials comprising starches are essentially cereals and legumes, such as corn, wheat, barley, sorghum, rice, potato, cassava or sweet potato, or algae.

Mention may in particular be made, among materials resulting from recovered materials, of plant or organic waste comprising sugars and/or starches.

Preferably, the renewable starting materials are plant materials.

The fermentation of the renewable materials takes place in the presence of one or more appropriate microorganisms; this microorganism may optionally have been modified naturally, by a chemical or physical stress, or genetically; the term used is then mutant. Conventionally, the microorganism used is Saccharomyces cerevisiae or one of its mutants.

Use may also be made, as renewable starting materials, of materials comprising cellulose or hemicellulose, indeed even lignin, which can be converted to sugar-comprising materials in the presence of the appropriate microorganisms. These renewable materials include straw, wood or paper. These materials can advantageously originate from recovered materials.

The lists presented above are not limiting.

Preferably, the fermentation stage is followed by a purification stage intended to separate the ethanol from the other alcohols.

The alcohol or alcohols obtained are dehydrated in stage b) in order to produce, in a first reactor, at least one alkene chosen from ethylene and mixtures of alkenes comprising ethylene, the byproduct from the dehydration being water.

Generally, the dehydration of the alcohol is carried out using a catalyst based on α-alumina, such as the catalyst sold by Eurosupport under the trade name ESM 110® (undoped trilobe alumina not comprising much residual Na₂O (approximately 0.04%)).

The operating conditions for the dehydration form part of the general knowledge of a person skilled in the art; by way of indication, the dehydration is generally carried out at a temperature of the order of 400° C.

Another advantage of the process according to the invention is its saving in energy: the fermentation and dehydration stages of the process according to the invention are carried out at relatively low temperatures of less than 500° C., preferably of less than 400° C.; in comparison, the stage of cracking and steam cracking oil to give ethylene is carried out at a temperature of the order of 800° C.

This saving in energy is also accompanied by a decrease in the level of CO₂ emitted to the atmosphere.

Preferably, a purification stage is carried out during stage a) or during stage b).

The optional stages of purification (purification of the alcohol(s) obtained in stage a), purification of the alkene(s) obtained in stage b)) are advantageously carried out by absorption on conventional filters, such as molecular sieves, zeolites, carbon black, and the like.

If the alcohol obtained in stage a) was purified so as to isolate the ethanol, the alkene obtained in stage b) is ethylene.

If the alcohol obtained in stage a) was not purified, a mixture of alkenes comprising ethylene is obtained on conclusion of stage b).

Advantageously, at least one purification stage is carried out during stage a) and/or stage b) in order to obtain ethylene with a degree of purity sufficient to carry out a copolymerization.

Particularly preferably, the alcohol obtained in stage a) is purified so as to isolate the ethanol; consequently, the alkene obtained in stage b) is ethylene.

The main impurities present in the ethylene resulting from the dehydration of the ethanol are ethanol, propane and acetaldehyde.

Advantageously, the ethylene should be purified, that is to say that the ethanol, the propane and the acetaldehyde should be removed, in order to be able to easily copolymerize in stage c).

The ethylene, the ethanol, the propane and the acetaldehyde can be separated by carrying out one or more low-temperature distillations.

The boiling points of these compounds are as follows:

| Compound | Boiling point (° C.) |
|---|---|
| Ethylene | −103.7 |
| Propane | −42.1 |
| Acetaldehyde | 20.8 |
| Ethanol | 75.5 |

The ethylene, the ethanol, the propane and the acetaldehyde are cooled to approximately −105° C., preferably −103.7° C., and then distilled in order to extract the ethylene.

Another advantage of the process according to the present invention relates to the impurities. The impurities present in the ethylene resulting from the dehydration of the ethanol are completely different from those present in the ethylene resulting from steam cracking. In particular, the impurities present in the ethylene resulting from steam cracking include dihydrogen and methane, this being the case whatever the composition of the initial feedstock.

Conventionally, dihydrogen and methane are separated after compressing to 36 bar and cooling to approximately −120° C. Under these conditions, the dihydrogen and the methane, which are liquids, are separated in the demethanizer and then the ethylene is recovered at 19 bar and −33° C.

The process according to the present patent application makes it possible to dispense with the stage of separation of the dihydrogen and methane and also makes it possible to cool the mixture to −105° C. at atmospheric pressure instead of −120° C. at 36 bar. The cooling of this separation stage can also take place under pressure in order to increase the boiling point of the compounds to be separated (for example approximately 20 bar and −35° C.). These differences also contribute to rendering the process according to the invention more economic (saving in equipment and saving in energy, which are also accompanied by a reduction in the level of CO₂ emitted to the atmosphere).

Another advantage is that the ethylene obtained in stage b) of the process according to the invention does not comprise acetylene, in contrast to the ethylene obtained by cracking or steam cracking. In point of fact, acetylene is highly reactive and brings about oligomerization side reactions; it is therefore particularly advantageous to obtain acetylene-free ethylene.

Another advantage is that the process according to the invention can be carried out in production units located on the site of production of the starting materials. In addition, the size of the production units of the process according to the invention is much smaller than the size of a refinery: specifically, refineries are large installations which are generally situated far from the centers of production of the starting materials and which are supplied via pipelines.

In stage c), the copolymerization of the monomers comprising the ethylene, the vinyl ester and optionally another comonomer, and an initiator of polymerization is carried out by polymerization in aqueous emulsion or by high-pressure polymerization in an autoclave or tubular reactor.

The high-pressure radical copolymerization is generally carried out by introducing the ethylene, the comonomers (carboxylic acid vinyl esters) and an initiator of polymerization at elevated pressure into an autoclave or tubular reactor at a temperature of between 80 and 325° C. in the tubular reactor and 150 to 290° C. in the autoclave reactor. The amount of the comonomers introduced can range up to 60% by weight, with respect to the total amount of the monomers (ethylene and comonomer) introduced into the reactor, which makes it possible to obtain a copolymer comprising up to 60% by weight of comonomer, for example carboxylic acid vinyl esters.

The pressure is regulated using a pressure-reducing valve situated at the outlet of the reactor. The polymer formed is recovered at the outlet of the reactor and the unreacted monomer is preferably recycled at the beginning of the reactor. The pressure inside the reactor is advantageously between 500 and 3000 bar and preferably between 1000 and 2500 bar.

Use may be made, with the comonomers and initiator, of a transfer agent; this transfer agent can, for example, be one or more alkanes, such as butane or pentane, one or more alkenes, such as propylene or butene, one or more aldehydes, such as propionaldehyde or acetaldehyde, or one or more ketones, such as acetone or methyl ethyl ketone. The molar mass of the polymer manufactured can be limited by adding this transfer agent.

Use may be made, as polymerization initiator, of any organic or inorganic compound which releases free radicals under the conditions of the reaction; preferably, use will be made of compounds or mixtures of compounds comprising a peroxide group, for example of the following compounds: tert-butyl peroxyneodecanoate, tert-butyl peroxypivalate, tert-amyl peroxypivalate, di(3,5,5-trimethylhexanoyl) peroxide, didecanoyl peroxide, tert-amyl peroxy-2-ethylhexanoate, tert-butyl peroxy-2-ethylhexanoate, tert-butyl peroxy-3,5,5-trimethylhexanoate, tert-amyl peroxy-3,5,5-trimethylhexanoate, tert-butyl peroxybenzoate, tert-butyl peroxyacetate or di(tert-amyl) peroxide.

Generally, the amount by weight of polymerization initiator is between 1 and 1000 ppm with respect to the total amount of the mixture introduced.

When a tubular reactor is used, the introduction of the mixture of ethylene and comonomers is preferably carried out at the top of the tubular reactor. The initiator or the mixture of initiators is injected, using a high-pressure pump, at the top of the reactor, after the point of introduction of the mixture of ethylene and comonomers.

The mixture of ethylene and comonomers can be injected at least at another location in the reactor; this injection is itself followed by a further injection of initiator or of a mixture of initiators; the term used is then multipoint injection technique. When the multipoint injection technique is used, the mixture is preferably injected in a way such that the ratio by weight of the mixture injected at the reactor inlet to the whole of the mixture injected is between 10 and 90%.

Other tubular high-pressure copolymerization processes which can be used are, for example, those described in US2006/0149004 A1 or US2007/0032614 A1

Use may also be made of an autoclave reactor for carrying out the high-pressure radical polymerization.

An autoclave reactor generally consists of a cylindrical reactor in which a stirrer is placed. The reactor can be separated into several zones connected to one another in series. Advantageously, the residence time in the reactor is between 30 and 120 seconds. Preferably, the length/diameter ratio of the reactor is between 3 and 25. The ethylene alone and the comonomer or comonomers are injected into the first zone of the reactor at a temperature of between 20 and 120° C., preferably between 50 and 80° C. An initiator is also injected into this first reaction zone. If the reactor is a multizone reactor, the stream of unreacted ethylene and comonomers and the polymer formed then pass into the following reaction zones. Ethylene, comonomers and initiators can be injected in each reaction zone. The temperature of the zones is between 150° C. and 290° C. and preferably between 160° C. and 280° C. The pressure of the reactor is between 500 and 3000 bar and preferably between 1200 and 2200 bar.

Other copolymerization processes which can be used are, for example, those described in the patent applications FR 2 660 660, FR 2 498 609, FR 2 569 411 and FR 2 569 412.

The emulsion polymerization makes possible the manufacture of copolymers comprising a content by weight of comonomers of between 40 and 99%. These copolymers can be copolymerized at low pressure, that is to say a pressure of less than 50 bar, the monomers being in emulsion in the water. Use may be made, for example, of the processes described in U.S. Pat. No. 7,189,461, U.S. Pat. No. 5,143,966 or U.S. Pat. No. 6,319,978.

A device which makes possible the implementation of the copolymerization process according to the invention according to a technique in which the reactants are injected at several points is presented in the single FIGURE appended as an annex.

This device comprises a tubular reactor R comprising five zones Z1, Z2, Z3, Z4 and Z5, a medium-pressure separator S1, which forms the inlet into a medium-pressure recycling circuit, and a low-pressure separator S2, which forms the inlet into a low-pressure recycling circuit.

The medium-pressure recycling circuit comprises the medium-pressure separator S1, the pipe 9 provided with the valve V4, the heat exchanger E7, the pipe 10, the separator S3 and the pipe 5 provided with the valve V6.

The low-pressure recycling circuit comprises the low-pressure separator S2, the pipe 14, the heat exchanger E8, the pipe 15, the separator S4, the pipe 17, the compressor C and the pipe 2.

The tubular reactor R is a tube comprising a jacket in which circulates water intended to contribute or remove heat for the purpose of heating or cooling the fluid moving through the reactor. The tubular reactor R comprises five zones Z1, Z2, Z3, Z4 and Z5 to which five parts of the jacket correspond: E1 is the part of the jacket situated around the zone Z1, E2 is the part of the jacket situated around the zone Z2, E3 is the part of the jacket situated around the zone Z3, E4 is the part of the jacket situated around the zone Z4 and E5 is the part of the jacket situated around the zone Z5; the flow rate and the temperature of the water circulating in each of the parts E1, E2, E3, E4 and E5 can be different.

According to this device, fresh ethylene moving through the pipe 1 (at a pressure of 60 bar) is admitted into the pipe 2 of the device. The pipe 1 is provided with a pressure-reducing valve V1.

In the pipe 2, this fresh ethylene is mixed with a gas stream (recycling of the ethylene and of the comonomer or comonomers of the low-pressure recycling circuit) originating from the compressor C.

The pipe 2 feeds the mixture to a precompressor Pc (where the mixture is compressed from 60 bar to 200 bar) and then the mixture exits from the precompressor Pc via the pipe 4. Fresh comonomers are introduced into the pipe 4 by means of the pipe 3. Downstream of the pipe 3, the pipe 5 introduces, into the pipe 4, the mixture of recycled fluids originating from the medium-pressure recycling circuit.

The mixture moving through the pipe 4 is introduced into the hypercompressor Hc (where the mixture is compressed from 200 bar to a pressure of between 1200 and 2500 bar, which is the pressure in the reactor) and then the mixture exits from the hypercompressor Pc via the pipe 6.

The pressure inside the reactor is regulated by the pressure-reducing valve V2.

If need be, the valve V7 makes it possible to regulate the pressure of the mixture in the pipe 6.

The reactor used comprises 5 zones: the mixture comprising the ethylene and the comonomer or comonomers and a transfer agent is admitted into the zone Z1 of the reactor (at the reactor inlet) by means of the pipe 6.

In the zone Z1, the mixture is heated up to the temperature of initiation of the polymerization reaction (between 90 and 170° C.).

At the inlet of the zone Z2, a mixture of ethylene and of comonomers can be introduced by means of the pipe M2 and a polymerization initiator (generally at least one peroxide and/or molecular oxygen) can be introduced by means of the pipe I2.

Then, in the zones Z3, Z4 and Z5, the pipes M3, M4 and M5 respectively make it possible to carry out additions of mixtures of ethylene and comonomers and the pipes I3, I4 and I5 respectively make it possible to carry out additions of polymerization initiator.

The polymerization reaction is highly exothermic and the temperature of the mixture which passes through the tubular reactor gradually increases.

A portion of the heat generated by the copolymerization reaction in the zones Z1, Z2, Z3, Z4 and Z5 is recovered by the water circulating in the corresponding part of the jacket E1, E2, E3, E4 and E5 respectively.

When additions by means of the pipes M2 to M5 and I2 to I5 are carried out in each zone Z1, Z2, Z3, Z4 and Z5, an identical temperature profile is obtained with a rise in the temperature up to a peak of between 180° C. and 325° C. (the beginning of the rise in temperature being due to the injection of the polymerization initiator and of the mixture) and then a decrease in the temperature (which corresponds to the end of the polymerization reaction and to the cooling of the stream by the jacket).

Before departing from the reactor, the mixture comprising the polymer is cooled to a temperature of between 140° C. and 240° C.

The mixture comprising the polymer exits from the reactor via the pipe 7 provided with a valve V3 which makes it possible to reduce the mixture in pressure, to a pressure of approximately 260 bar. The mixture then enters the heat exchanger E6, where it is cooled and departs therefrom via the pipe 8.

The pipe 8 conveys the mixture to the medium-pressure separator S1. In S1, the copolymer formed is separated from the mixture of the unreacted products: ethylene, comonomers and transfer agent.

The mixture of the unreacted products is conveyed to the heat exchanger E7 via the pipe 9, where it is cooled and then exits from the heat exchanger E7 via the pipe 10. The pipe 10 conveys the products to the separator S3, where the polymer waxes (having a low weight and which were not separated in the separator 51) are isolated and extracted from the device via the pipe 11. The pipe 5, provided with a valve V6, conveys the mixture of ethylene, comonomers and transfer agent from the separator S3 to the pipe 4.

The copolymer exits from the separator S1 via the pipe 12 provided with a valve V5 and is introduced into the low-pressure separator S2. The valve V5 makes it possible to reduce the copolymer in pressure to a pressure of approximately 2 to 5 bar. The copolymer is extracted from the device via the pipe 13 and then it is sent to an extruder in order to be converted into granules.

The pipe 14 makes it possible to discharge the gas mixture (ethylene/comonomers, transfer agent which were not separated in the separator S1); this mixture is conveyed to the heat exchanger E8 via the pipe 14, where it is cooled down to approximately 35° C. and then exits from the heat exchanger E8 via the pipe 15. The pipe 15 conveys the products to the separator S4, where the monomers are condensed. A portion of the monomers is extracted from the device via the pipe 16 and the other portion of the monomers is introduced via the pipe 17 into the compressor C (where they are compressed at 60 bar).

The monomers exit from the compressor via the pipe 2. The pipe 18 makes it possible to introduce the transfer agents into the pipe 2.

In stage d), the copolymer obtained is isolated and optionally purified, according to a conventional technique, as a function of the application for which it is intended.

According to a specific alternative form, use may be made of the vinyl ester, and in particular the vinyl acetate and the vinyl propionate comprising carbon atoms of renewable origin.

These vinyl esters can be obtained according to the processes described in the application FR0854976 of the applicant company.

The vinyl acetate is capable of being obtained according to the process comprising the following stages:
a) fermentation of renewable starting materials and optionally purification, in order to produce at least one alcohol chosen from ethanol and mixtures of alcohols comprising ethanol;
b) separation of the alcohol obtained into two parts, then the introduction of the first part into a first reactor and of the second part into a second reactor;
c) in the first reactor, dehydration of the alcohol obtained in order to produce at least one alkene chosen from ethylene and mixtures of alkenes comprising ethylene and optionally purification of the ethylene;
d) in the second reactor, oxidation of the alcohol obtained in order to produce acetic acid and optionally purification of the acetic acid;
e) introduction, into a third reactor, of the ethylene obtained on conclusion of stage c) and of the acetic acid obtained on conclusion of stage d) and implementation of the reaction for the acyloxylation of the ethylene;
f) isolation and optionally purification of the vinyl acetate obtained on conclusion of stage e);
   and the vinyl propionate is capable of being obtained according to the process comprising the following stages:
a. fermentation of renewable starting materials and optionally purification, in order to produce at least one alcohol chosen from ethanol and mixtures of alcohols comprising ethanol;
b. dehydration of the alcohol obtained in order to produce, in a first reactor, at least one alkene chosen from ethylene and mixtures of alkenes comprising ethylene and optionally purification of the ethylene;
c. dehydration of glycerol in a second reactor, in the presence of molecular oxygen, in order to produce acrylic acid;
d. hydrogenation of the acrylic acid in the presence of molecular oxygen in order to produce propanoic acid;
e. introduction, into a third reactor, of the ethylene obtained on conclusion of stage b) and of the propanoic acid obtained on conclusion of stage d) and implementation of the reaction for the acyloxylation of the ethylene;
f. isolation and optionally purification of the vinyl propionate obtained on conclusion of stage e).

The ethylene used in step c) of the process for the manufacture of vinyl acetate and used in stage b) of the process for the manufacture of vinyl propionate is then obtained in the same advantageous manner as the ethylene used in the comonomers according to the present invention.

The process for the manufacture of the terpolymers of ethylene, of at least one vinyl ester and of at least one unsaturated carboxylic acid anhydride according to the present patent application employs, in stage c), a reaction for the copolymerization of the ethylene with at least one vinyl ester, advantageously vinyl acetate or vinyl propionate, and at least one unsaturated carboxylic acid anhydride, advantageously maleic anhydride. This stage is carried out in the same way as stage c) for the manufacture of copolymers of ethylene and of at least one vinyl ester.

According to a specific alternative form, use may be made of a vinyl ester and/or of maleic anhydride comprising carbon atoms of renewable origin.

These vinyl esters can be obtained according to the processes mentioned above and described in the application FR0854976 of the applicant company.

The maleic anhydride can be obtained according to the process described in the application FR 0 854 896 of the applicant company, comprising the following stages:
a) fermentation of renewable starting materials and optionally purification in order to produce a mixture comprising at least butanol;
b) oxidation of the butanol to give maleic anhydride at a temperature generally of between 300 and 600° C., using a catalyst based on vanadium and/or molybdenum oxide;
   isolation of the maleic anhydride obtained on conclusion of stage b).

Preferably, the copolymers according to the invention are chosen from:
random copolymers of ethylene and of at least one vinyl ester, in particular random copolymers of ethylene and of vinyl acetate and random copolymers of ethylene and of vinyl propionate;
random terpolymers of ethylene, of at least one vinyl ester and of maleic anhydride, in particular random terpolymers of ethylene, of vinyl acetate and of maleic anhydride and random copolymers of ethylene, of vinyl propionate and of maleic anhydride;
terpolymers of ethylene, of vinyl acetate and of a carboxylic acid (meth)acrylic ester;
random copolymers of ethylene and of at least one vinyl ester which are grafted with an unsaturated carboxylic acid anhydride, in particular random copolymers of ethylene and of vinyl acetate which are grafted with maleic anhydride.

Advantageously, the unsaturated carboxylic acid esters used in the terpolymers according to the invention are alkyl (meth)acrylates; the number of carbon atoms of the alkyl part of the alkyl (meth)acrylates preferably ranges from 1 to 24; in particular, the alkyl (meth)acrylates are chosen from methyl acrylate, ethyl acrylate, n-butyl acrylate, isobutyl acrylate or 2-ethylhexyl acrylate. Particularly preferably, use is made of methyl acrylate, n-butyl acrylate and 2-ethylhexyl acrylate.

The copolymers according to the invention preferably comprise from 40 to 99% of ethylene by weight and from 1 to 60% of carboxylic acid vinyl ester; more preferably, from 55 to 90% of ethylene by weight and from 10 to 45% of carboxylic acid vinyl ester.

The terpolymers according to the invention preferably comprise from 40 to 99% of ethylene by weight, from 0.99 to 50% of carboxylic acid vinyl ester by weight and from 0.01 to 10% of maleic anhydride or (meth)acrylic ester; more preferably, from 60 to 94.95% of ethylene by weight, from 5 to 35% of carboxylic acid vinyl ester by weight and from 0.05 to 5% of maleic anhydride or (meth)acrylic ester.

The melt flow index MFI of these copolymers is advantageously within the range extending from 0.1 to 1000 g/10 min (ASTM D 1238, 190° C., 2.16 kg), preferably from 1 to 500 g/10 min.

The copolymer or the terpolymer can be amorphous or semicrystalline. When it is semicrystalline, its melting point can be between 45° C. and 115° C.

According to a specific form of the invention, the copolymer is partially or completely saponified, that is to say that the vinyl ester functional group of the copolymer is hydrolyzed in order to form an alcohol functional group. This saponification can be carried out by the techniques known to a person skilled in the art. On carrying out the saponification of the ethylene/vinyl acetate (EVA) copolymer, a saponified ethylene/vinyl acetate (EVOH) copolymer is obtained, that is to say that at least a portion of the vinyl acetate functional groups of the copolymer react to form vinyl alcohol.

These saponified copolymers have excellent barrier properties to gases, allowing them to be advantageously used in multilayer structures, in particular in food packaging.

The invention also relates to compositions comprising, in addition to the copolymer or the terpolymer, at least one additive for improving the properties of the final material.

These additives include antioxidants; UV protecting agents; “processing” aids having the role of improving the appearance of the final polymer during the processing thereof, such as fatty amides, stearic acid and its salts, ethylenebisstearamide or fluoropolymers; defogging agents; antiblocking agents, such as silica or talc; fillers, such as calcium carbonate and nanofillers, such as, for example, clays; coupling agents, such as silanes; crosslinking agents, such as peroxides; antistatic agents; nucleating agents, pigments; or dyes. These additives are generally used in contents of between 10 ppm and 100 000 ppm by weight, with respect to the weight of the final copolymer. The compositions can also comprise additives chosen from plasticizers, viscosity reducers or flame-retardant additives, such as aluminum or magnesium hydroxides (the latter additives can reach amounts far above 100 000 ppm). Some of these additives can be introduced into the composition in the form of masterbatches. These compositions can also comprise other polymers, such as polyolefins other than the copolymers according to the invention, polyamide or polyester.

Mention may be made, as examples of polyolefins other than the polymer according to the invention, of homopolymers and copolymers of ethylene, such as very low density polyethylene (VLDPE), low density polyethylene (LDPE), linear low density polyethylene (LLDPE), medium density polyethylene (MDPE), high density polyethylene (HDPE), copolymers comprising ethylene and vinyl acetate or copolymers comprising ethylene and alkyl (meth)acrylate, the ethylene of which does not result from renewable starting materials.

It is also possible to form films from the copolymers, for example encapsulating films for solar panels, agricultural films, packaging films, thermo-adhesive films, protective films.

The present application is also targeted at the uses of the copolymers according to the invention and compositions comprising at least one copolymer according to the invention, in particular the uses of the copolymers according to the invention as adhesives or as adhesive compositions in a multilayer structure.

The present application is targeted in particular at the uses of the copolymers and compositions according to the invention as adhesives or adhesive compositions, in particular in coextrusion, in extrusion-coating or in extrusion-lamination. These copolymers according to the invention exhibit an adhesion to numerous supports, such as metals or polymers, for example polyesters, polyamides, polyolefins or polymers which exhibit barrier properties towards water, gases or hydrocarbons.

Mention may be made, in coextrusion, among the coextrudable supports, of all types of polymers such as polyethylene terephthalate (PET), polypropylene (PP), polyvinyl chloride (PVC), polyvinylidene chloride (PVDC), polyvinyl fluoride (PVF), polyvinylidene fluoride (PVDF), polyamide (PA), polystyrene (PS), and the like. The adhesives and adhesive compositions can thus be used in multilayer structures, in particular between a layer of polyethylene terephthalate (PET) and a layer of polyethylene or between a layer of polyester resulting from renewable materials, such as, for example, polylactic acid), and a polymer having barrier properties, such as, for example, the saponified ethylene/vinyl acetate (EVOH) copolymer or PA, which have barrier properties towards oxygen.

Thus, the invention relates to a multilayer structure obtained by use of the adhesive composition according to the invention in an extrusion-coating process for application to a support, said support being chosen from aluminum, paper or board, cellophane, films based on polyethylene, polypropylene, polyamide, polyester, polyvinyl chloride (PVC), polyvinylidene chloride (PVDC) or polyacrylonitrile (PAN) resins, these films being or not being orientated, being or not being metalized and being or not being treated by a physical or chemical route, and films coated with a thin inorganic barrier layer, such as polyester (PET SiOₓ or AlOₓ).

The invention also relates to a multilayer structure obtained by use of the adhesive composition of the invention in an extrusion-lamination process for adhesively bonding several supports together, which supports are different in nature; the supports are generally chosen from aluminum, paper or board, cellophane, films based on polyethylene, polypropylene, polyamide, polyester, polyvinyl chloride (PVC), polyvinylidene chloride (PVDC) or polyacrylonitrile (PAN) resins, these films being or not being orientated, being or not being metalized and being or not being treated by a physical or chemical route, and films coated with a thin inorganic barrier layer, such as polyester (PET SiOₓ or AlOₓ).

The present application is also targeted at the use of the compositions according to the invention as sealing layer, in particular over a material chosen from aluminum, polystyrenes (PS), polypropylenes (PP), polyamines (PA), and the like.

The copolymers according to the invention can also be used:
as compatibilizing agent for compounds (that is to say, an adjuvant which makes it possible to improve the compatibility with said compounds), in particular natural fibers, polyolefin/polyamine (PO/PA) alloys, polyolefin/polyester or biopolyester alloys, polyolefin/starch alloys, and the like,
as impact modifier in polymers (that is to say, as adjuvant in a polymer which makes it possible to improve the impact strength of said polymer), such as polyethylene terephthalate (PET) polymers, polyamides (PA), polypropylenes (PP), polybutylene terephthalates (PBT) or biopolyesters (polylactic acid (PLA), polyhydroxyalkanoates (PHA), and the like).

It is also possible to form films from the copolymers, such as encapsulating films for solar panels. A preferred composition for the manufacture of an encapsulating film comprises a mixture of a random copolymer of ethylene and of vinyl acetate with a random terpolymer of ethylene, of vinyl acetate and of maleic anhydride, this copolymer and/or this terpolymer being according to the invention. It is also possible to form flexible coverings, in particular for the construction industry, for the floor or the walls, or in the automobile industry, agricultural films, thermo-adhesive films, protective films or packaging films.

Use may be made of the copolymers and compositions according to the invention as additives in oil or fuels. These copolymers can also participate in the composition of an ink. In these applications, the ethylene/vinyl ester/carboxylic acid (meth)acrylic ester copolymers are particularly advantageous.

These copolymers can also be used to manufacture a soundproofing body, that is to say a crosslinkable expandable body having a soundproofing function. Flexible components can also be formed from the copolymers of the invention by injection or thermoforming; it is also possible to manufacture pipes or containers, such as bottles or tanks, by tube extrusion or by blow molding.

The copolymers according to the invention can also be present in compositions for manufacturing woven or nonwoven textiles.

Another possible application for the copolymers according to the invention is that of manufacturing masterbatches. It is also possible to use the copolymers according to the invention to manufacture electric cables. In particular, they can be used to manufacture an electric cable sheath. It is also possible to manufacture compositions by dispersing a conducting compound (for example carbon black) in order to form half-conducting half-insulating compositions (commonly known as semi-conducting compositions); these compositions are of particular use in the manufacture of medium- or high-voltage cables.

These copolymers can also be used as an asphalt modifier. The copolymer according to the invention can also participate in the composition of a hot-melt adhesive.

In particular, a hot-melt adhesive composition can be formulated by mixing “tackifying” resins, waxes and antioxidants with the copolymers according to the invention. It is also possible to add other additives thereto, such as plasticizers, viscosity reducers, pigments or fillers.

The “tackifying” resins can be solid or liquid and can be used alone or as a mixture; they make it possible mainly to contribute adhesiveness to the composition. Mention may be made, among them, of:
resins based on natural or modified, for example polymerized, rosin resin, in particular pentaerythritol or glycerol esters, terpene or polyterpene resins which are modified,
synthetic resins of α-methylstyrene, styrene/terpene or terpene/phenol type,
resins of hydrocarbon origins, such as aliphatic or aromatic resins which are nonhydrogenated or completely or partially hydrogenated,
waxes, which make it possible to adjust the fluidity and the setting time of the adhesive, can be chosen from the following families: paraffin waxes, microcrystalline waxes, polyethylene waxes, oxidized or nonoxidized Fischer-Tropsch waxes, or functionalized waxes of hydroxystearamide or fatty amide type.

### EXAMPLES

A copolymer of ethylene and of vinyl acetate according to the present invention was prepared from ethylene obtained by employing stages a) and b) according to the process of the present application and by then carrying out a copolymerization (stage c)) using the device described above and presented in the single FIGURE appended as an annex. The tubular reactor used measures 600 m in length and 42 mm in diameter. The ethylene is injected at a flow rate of 12 tonnes/hour (pipe 1) and vinyl acetate is injected at a flow rate of 800 kg/hour (pipe 3); the mixture is compressed in the hypercompressor (He) to 2400 bar. The mixture is preheated to 120° C. in the zone Z1 and then a Lup 11/26 mixture (that is to say, Luperox 11, tert-butyl peroxypivalate/Luperox 26, tert-butyl peroxy-2-ethylhexanoate) is injected via the pipe I2. In the zone Z2, the temperature rises up to 210° C. and then falls again to 160° C. at the outlet of zone Z2. A Lup 11/26 mixture is then reinjected in each zone Z3, Z4 and Z5, at the inlet of the zone; the temperature rises up to 210° C. and then falls again to 160° C. at the outlet of the zone. 1.7 tonnes/hour of ethylene/vinyl acetate copolymer, with a vinyl acetate content of 6% by weight and a melt flow index of 0.5, are obtained.

1. A copolymer of ethylene and of at least one carboxylic acid vinyl ester, in which the ethylene is at least partially obtained from renewable starting materials.

2. The copolymer as claimed in claim 1, in which at least a portion of the carbon atoms of the carboxylic acid vinyl ester is of renewable origin.

3. The copolymer as claimed in claim 1, wherein said copolymer is a terpolymer of ethylene, of at least one carboxylic acid vinyl ester and of at least one unsaturated carboxylic acid anhydride, in which optionally at least a portion of the carbon atoms of the unsaturated carboxylic acid anhydride are of renewable origin.

4. The copolymer as claimed in claim 1, wherein said copolymer is a terpolymer of ethylene, of at least one vinyl ester and of at least one unsaturated carboxylic acid ester, in which optionally at least a portion of the carbon atoms of the unsaturated carboxylic acid ester are of renewable origin.

5. The copolymer as claimed in claim 1, wherein said copolymer is chosen from:
random copolymers of ethylene and of at least one carboxylic acid vinyl ester;
random terpolymers of ethylene, of at least one carboxylic acid vinyl ester and of maleic anhydride;
terpolymers of ethylene, of vinyl acetate and of a carboxylic acid (meth)acrylic ester;
random copolymers of ethylene and of at least one vinyl ester which are grafted with an unsaturated carboxylic acid anhydride.

6. The copolymer as claimed in claim 1, wherein said copolymer is partially or completely saponified.

7. The copolymer as claimed in claim 1, wherein said copolymer comprises at least 0.24×10⁻¹⁰% by weight of ¹⁴C, this amount of ¹⁴C being determined by one of the methods described in the standard ASTM D6866-06.

8. A process for the manufacture of a copolymer of ethylene and of at least one carboxylic acid vinyl ester as claimed in claim 1, comprising the following stages:
a) fermenting renewable starting materials and optionally purification, in order to produce at least one alcohol chosen from ethanol and mixtures of alcohols comprising ethanol;
b) dehydrating the alcohol obtained in order to produce at least one alkene chosen from ethylene and mixtures of alkenes comprising ethylene and optionally purification of the alkene in order to obtain ethylene,
c) copolymerizing the ethylene with at least one comonomer chosen from carboxylic acid vinyl esters,
d) isolating the copolymer obtained.

9. A process for the manufacture of a terpolymer of ethylene, of at least one vinyl ester and of unsaturated carboxylic acid anhydride as claimed in claim 3, comprising the following stages:
a) fermenting renewable starting materials and optionally purification, in order to produce at least one alcohol chosen from ethanol and mixtures of alcohols comprising ethanol;
b) dehydrating the alcohol obtained in order to produce at least one alkene chosen from ethylene and mixtures of alkenes comprising ethylene and optionally purification of the alkene in order to obtain ethylene;
c) copolymerizing the ethylene with at least one vinyl ester and at least one unsaturated carboxylic acid anhydride;
d) isolating the copolymer obtained.

10. The process for the manufacture of a copolymer as claimed in claim 8, wherein the renewable starting materials are plant materials selected from the group consisting of sugar cane and sugar beet, maple, date palm, sugar palm, sorghum, American agave, corn, wheat, barley, sorghum, rice, potato, cassava, sweet potato, algae, materials comprising cellulose, materials comprising hemicelluloses, wood, straw and paper.

11. The process for the manufacture of a copolymer as claimed in claim 8, wherein the copolymerization of stage c) is a high-pressure radical copolymerization carried out by introducing the ethylene, the comonomer or comonomers and an initiator of polymerization at elevated pressure either into a tubular reactor at a temperature of between 80 and 325° C., or into an autoclave reactor at a temperature of between 150 and 290° C., the amount of the comonomers introduced into the tubular or autoclave reactor ranging up to 60% by weight, with respect to the total amount of ethylene and comonomers introduced into the reactor.

12. The process for the manufacture of a copolymer as claimed in claim 11, wherein the copolymerization of stage c) is carried out in a tubular reactor, the mixture of the ethylene and the comonomers being injected at least at another location in the reactor, this injection being itself followed by a further injection of initiator or of mixture of initiators.

13. A composition comprising a copolymer as claimed in claim 1, wherein said copolymer additionally contains at least one additive chosen from antioxidants; UV protection agents; processing aids; defogging agents; antiblocking agents; fillers; coupling agents; crosslinking agents; antistatic agents; nucleating agents; pigments; dyes; plasticizers; viscosity reducers; or flame-retardant additives.

14. The copolymer as claimed in claim 1, comprising an adhesive or adhesive composition, in particular in coextrusion, in extrusion-coating or in extrusion-lamination.

15. (canceled)

16. The copolymer as claimed in claim 1, comprising a compatibilizing agent for compounds or as an impact modifier in polymers.

17. The use of a copolymer as claimed in claim 1, comprising encapsulating films for solar panels, flexible coverings, an additive in petroleum or in fuels; a soundproofing body, flexible components, pipes, bottles, electric cable sheaths, an ink, woven, nonwoven textiles, asphalt or hot-melt adhesives.

18. The copolymer as claimed in claim 5, wherein
said random copolymers of ethylene and of at least one carboxylic acid vinyl ester, are selected from the group consisting of random copolymers of ethylene and of vinyl acetate, and random copolymers of ethylene and of vinyl propionate;
said random terpolymers of ethylene, of at least one carboxylic acid vinyl ester and of maleic anhydride, are selected from the group consisting of random terpolymers of ethylene, of vinyl acetate and of maleic anhydride and random terpolymers of ethylene, of vinyl propionate and of maleic anhydride;
said random copolymers of ethylene and of at least one vinyl ester which are grafted with an unsaturated carboxylic acid anhydride, are selected from the group consisting of random copolymers of ethylene and of vinyl acetate which are grafted with maleic anhydride.

19. The process for the manufacture of a copolymer as claimed in claim 9 wherein the renewable starting materials are plant materials selected from the group consisting of sugar cane and sugar beet, maple, date palm, sugar palm, sorghum, American agave, corn, wheat, barley, sorghum, rice, potato, cassava, sweet potato, algae, materials comprising cellulose, materials comprising hemicelluloses, wood, straw and paper.
